(19) United States
(12) Patent Application Publication
(10) Pub. No.: US 2005/0265518 A1
(43) Pub. Date: Dec. 1, 2005

(54) Mammography procedure and apparatus for reducing pain when compressing a breast

(76) Inventor: Leo J. Aubel, Lincolnshire, IL (US)

Correspondence Address: Leo J. Aubel, 111 Revershire Lane, Lincolnshire, IL, 60069, US

(21) Appl. No.: 11/145,400

(22) Filed: Jun. 3, 2005

Related U.S. Application Data
(63) Continuation-in-part of application No. 10/789,001, filed on Feb. 26, 2004, now abandoned.
(60) Provisional application No. 60/534,603, filed on Jan. 6, 2004.

(51) Int. Cl. A61B 6/04

(52) U.S. Cl. 378/37

(57) ABSTRACT

A method and apparatus for compressing a patient's breast when using an X-ray mammography machine to take an image wherein said machine has a compression paddle and a bucky. A movable interface plate is mounted on the bucky as an interface between the bucky and a patient's breast. The method includes a step wherein the compression paddle is moved downwardly to provide compression forces on the breast; the movement of the compression paddle is stopped at a position where less than the full desired compression of the breast is attained. Next, the movable interface plate is elevated upwardly against the breast to obtain the full desired compression. The upward movement of the interface plate functions to distribute and balance the compression and shear forces applied to the breast.

This application claims the priority filing date of U.S. provisional application Ser. No. 60/534,603 filed on Jan. 6, 2004 by the applicant herein. This application is a continuation-in-part of Ser. No. 10/789,001 filed on Feb. 26, 2004.

### BACKGROUND OF INVENTION

The invention refers to a new method and apparatus used when compressing a patient's breast for taking a radiographic image. In mammography, compression of the breast is essential for good quality X-ray imaging. Firm compression spreads out the breast tissue, thereby reducing superimposed structures. Likewise, the low dosage X-rays used in mammography can more easily penetrate the thinner mass that results from compression and spreading of the breast.

Women are advised to undergo periodic mammography screening (examination) in an attempt to detect cancer at its earliest stages. Unfortunately, because of the harsh breast compression techniques, patients consider mammograms to be uncomfortable, even painful, and studies show that patients are reluctant to schedule screenings after their first experience with the procedure.

A variety of methods have been tried in an effort to ease the patient's discomfort. One method is to allow the patient to control some facet of the breast compressive forces. Another method is to train the technician to be more empathetic to each particular patient's demeanor. Still another method is to provide a breast cushion interface plate as disclosed in U.S. Pat. Nos. 5,185,776 and 5,377,254 and 6,577,702. A unique type of machine for improving the mammography procedure was disclosed in U.S. Pat. No. 5,590,166 wherein the bucky (the support and container for the X-ray film or digital imaging device) and the breast compression paddle, can move toward each other “simultaneously at a substantially equal but oppositely directed speed” for compressing the breast.

The present invention is directed to the same important purpose: making the mammography procedure less painful. The inventive method requires minimal additional accessories to the mammography machine equipment, and requires minimal additional training or experience in utilizing the method. Further, the quality of the images obtained by the digital detectors or film of the X-ray machine are improved since, with the new compression technique, the mass of the breast will tend to be spread out somewhat more uniformly than in prior art procedures. Most importantly, the unique breast interface and compression interface plate disclosed herein will be more comfortable for the patient.

### SUMMARY OF THE INVENTION

A method and apparatus are disclosed wherein a movable breast interface element mounted on the bucky is selectively actuated and moved toward the associated compression paddle during the breast compression procedure. This provides cooperating, more uniform, more evenly distributed, and more comfortable compressive and shear forces to the breast.

The foregoing features and advantages of the present invention will be apparent from the following more particular description of the invention. The accompanying drawings, listed herein below, are useful in explaining the invention.

### DRAWINGS

FIG. 1 is a sketch showing the inventive interface assembly mounted on a standard mammography machine;

FIG. 2 is a sketch, labeled prior art, showing a mammography machine as disclosed in U.S. Pat. No. 5,590,166 wherein the bucky is movable on the C-arm;

FIG. 3, labeled prior art, is a sketch indicating a compression paddle compressing a patient's breast a desired amount;

FIG. 4 is a sketch showing the inventive apparatus and concept of a movable breast interface plate mounted on the bucky;

FIG. 5 is a sketch showing the position of the plate on the bucky lifting the breast toward the compression paddle in accordance with the inventive method;

FIG. 6 shows a sketch of a breast useful in explaining the method of the invention.

FIG. 7 is a sketch depicting, by the arrow line, the compression and shear forces on a patient's breast as in prior methods;

FIG. 8 depicts the complementary and cooperating compression forces developed by moving (elevating) the plate on the bucky, in accordance with the inventive method;

FIG. 9 depicts one embodiment of a breast interface element in accordance with the invention;

FIG. 10 shows a view of the interface element of FIG. 9 in an expanded mode;

FIG. 11 shows a view of the interface element of FIG. 9 mounted on the bucky and in an expanded mode;

FIG. 12 shows a view of FIG. 10 in partial cross section to more clearly show an expanded configuration of the interface element;

FIG. 13 shows an embodiment of the interface element wherein a portion of the top surface is contoured to cradle a patient's breast;

FIG. 14 shows an embodiment of the breast interface assembly wherein linear actuators are positioned to elevate or raise a breast interface element comprising a plate;

FIG. 15 shows a relative enlarged view of the mounting of a linear actuator on the bucky;

FIG. 16 is a front view of the interface element comprising a plate of FIG. 14 when the plate is in a first lowered mode contiguous to the bucky;

FIG. 17 is a front view of the interface plate of FIG. 14 in an elevated mode raised upwardly from the bucky; and

FIG. 18 shows a linear actuator mounting, wherein a narrow strip is provided to support the linear actuator adjacent to the bucky.

### DESCRIPTION OF THE INVENTION

FIG. 1 depicts a standard mammography machine 10. The machine 10 includes a C-arm 11 mounted on a base 12. An X-ray source 13 mounted on the upper end of the C-arm 11 selectively provides an X-ray beam directed toward a bucky 15 that is supported at the lower end of the C-arm. The bucky is fixed or stationary relative to the C-arm.

The C-arm, including the bucky, is rotatable or tiltable for, e.g., taking oblique images of the breast. Contained within the bucky 15 is a suitable known type of image detecting and recording device 19 (such as a digital image detector or a film cassette) that is inserted through a slot 19A into the bucky, see FIG. 3.

The patient is depicted by the dotted lines of FIG. 1. The bucky 15 is oriented to provide a support bench or table for the patient's breast during the mammography procedure.

While the bucky is stationary or fixed relative to the C-arm, a breast compression paddle 14, mounted on the center or bight of the C-arm, is movable relatively toward and away from the bucky 15. The compression paddle 14 includes a bottom surface that is operated to push and compress the patient's breast between the paddle 14 and the bucky 15, as depicted in FIG. 3.

The present invention discloses a method and apparatus for compressing the breast during the mammography procedure wherein a breast interface element mounted on the bucky is controllably movable relative to the bucky.

In the prior art, a first step in the mammography procedure for taking a craniocaudal (head-to-toe orientation) image is to position the patient's breast 16 on the bucky 15 such that the weight (mass) of the breast is supported on the bucky. As depicted in FIG. 3, the next step is to firm the compression paddle 14 against the chest wall, the upper surface of the breast and the suspensory ligaments of the breast. Next, the compression paddle 14 is powered to move down substantially parallel to and adjacent to the chest wall, engaging the upper posterior portion of the breast, hence pushing and forcing the breast downward.

As labeled in FIG. 3, in order to provide a desired spacing, indicated as “X”, between the compression paddle 14 and bucky 15 the breast is compressed to desired position for taking an X-ray image. This develops compression forces and shear forces on the chest wall and the breast. The human skin does not stretch easily, and depending on the size of the breast and the condition of the muscles and ligaments, the shear forces applied can be substantial as the compression paddle 14 is forced down to compress the breast to a desired position. Many patients complain that this method is quite painful.

In the inventive method, and as depicted in FIG. 4, the breast is positioned on a breast interface element 30 positioned on the bucky and the compression paddle 14 is moved to compress the breast, as in previous methods. Importantly however, in the inventive method, before the desired selected compression of the breast is attained, the movement of the compression paddle 14 is paused at some intermediate position where the compression force has compressed the breast to less than the full desired compression. Next, and as depicted in FIG. 5, breast interface element 30 mounted on the bucky 15 is moved toward the paddle 14 to obtain the full desired compression.

FIGS. 4 and 5, graphically show the inventive method. A majority, but less than the full desired compression, is indicated in FIG. 4 as “X+Y” cm. It should be understood that because of the different sizes, configurations and firmness of patients' breasts both “X” and “Y” are variables. In use, the technician determines the required compression for each individual breast. At the intermediate position, it is approximated that 70% to 85% of the desired compression has been attained; these indicated percentages of compression are not preset, but rather the technician determines the amount of compression through training and experience based on the required compression for proper imaging, while also taking into consideration the patient's comfort.

Patients usually appear to sense a higher degree of pain at the higher compressive forces when the breast is compressed to the full desired compression. Patients appear to sense much lesser pain or discomfort at an intermediate position of the sequence. However, at the intermediate position of the paddle 14 (as indicated in FIG. 4) the breast has not been sufficiently compressed for taking the X-ray image. Accordingly, in the next step of the inventive procedure, movement of the paddle 14 is paused at the aforementioned intermediate position, and the paddle now becomes a fixed upper support against which the breast is compressed by the interface element 30. In the following step, depicted in FIG. 5, the breast interface element 30 on the bucky 15 is caused to move upwardly from its initial position (indicated by the dotted lines) to the “full” position indicated by the solid lines, to compress the breast there between. The breast interface element 30 on the bucky 15 is moved toward the compression paddle 14 a distance of “Y” cm, an amount equal to the amount necessary to compress the breast to a position to provide the desired separation “X”. The breast is now in position for taking an X-ray image.

For additional description, refer to FIG. 6 that shows an outline of a patient's breast 16. Essentially, in the latter steps of the inventive method, the initial roles of the compression paddle 14 and the bucky 15 are effectively reversed. That is, the compression paddle 14 becomes a base reference surface and the breast interface element 30 on the bucky 15 is moved to apply the additional compressive force to the sagittal section 20 (the underside) of the breast.

Refer now also to FIGS. 7 and 8. The inventive method tends to decrease the compressive and shear forces applied to the upper section of the breast including the major pectoral muscles and suspensory ligaments of the breast, and in so doing reduces the pain felt by the patient. Note that as depicted in FIG. 6, the sagittal section 20 of the breast is normally lower than the inframammary fold 21 that joins the sagittal section to the chest wall. As depicted in FIG. 7, when the breast is positioned on the breast interface element 30, the inframammary fold 21 and the sagittal section 20 are essentially level with the upper surface of the bucky.

In the prior methods, when the compression paddle 14 is lowered toward the bucky 15, essentially all the compressive and shear forces are applied to the top of the breast; that is, the forces are effective on the suspensory ligaments, tissue and muscles of the breast, see FIG. 7. There are limited compressive and shear forces on the sagittal section 20 of the breast and on the tissue and ligaments adjacent the inframammary fold 21.

The inventive method provides a procedure for distributing the compressive and shear forces applied to the breast. More specifically, in the inventive method after the compression paddle 14 is paused at an intermediate position, the breast interface element 30 on the bucky 15 is caused to move up to provide an active compression force to the sagittal section 20 of the breast. This is indicated in FIG. 8 by the arrow line and labeled “complementary and cooperating compression forces”. Upward movement of the breast interface element compensates for the distance that the compression paddle 14 would have moved in the prior methods.

As depicted in FIG. 8, compression and shear forces will still be applied to the top muscles and ligaments of the breast which will still tend to be stretched, but much more moderately. Note that as the breast interface element 30 on the bucky 15 is caused to move up, the tissue and ligaments on the sagittal section 20 and the adjacent chest wall will also be subjected to shearing and compressive forces. However, the level of these shearing and compressive forces will be more distributed and balanced. Likewise, the forces applied to the upper posterior of the breast will be substantially less than the forces applied by the prior art. As stated above, since the size and types of patients' breast vary so considerably, the actual distance of movement of the breast interface element 30 on the bucky 15 varies.

As further explanation, the breast may be considered as a non-symmetrical object effectively suspended from a vertical surface. Muscle, ligaments and tissue that extend from the chest wall above the breast principally suspend the breast, see FIG. 6. In prior mammography compression procedures, the sagittal section of the breast has been “passively” supported on the bucky, and all the compressive forces have been applied to the upper posterior section of the breast, see FIG. 7. In the prior art, as the compression paddle is moved to compress the breast, the edge of the paddle engages the upper posterior section of the breast at an angle, and the compressive and shear forces that are developed by the paddle will tend to stretch the muscle, ligaments and tissue of the upper posterior of the breast while minimal forces effect the sagittal section of the breast. As the compression paddle is moved, the shear forces tend to cause stretching of the muscles, ligaments and tissue suspending the breast, which is a major source of the pain experienced by the patient. During this same period of paddle movement, other portions of the breast including the sagittal section may only be minimally stressed.

In contrast to the prior art, in the present invention a basic principle is to more evenly distribute the forces applied to the breast during the compression procedure. This reduces the shear forces applied to the breast suspending muscles, ligaments and tissue and applies additional compressive forces to other parts of the breast. The total effective compression force on the breast remains essentially the same, but it is more uniformly distributed.

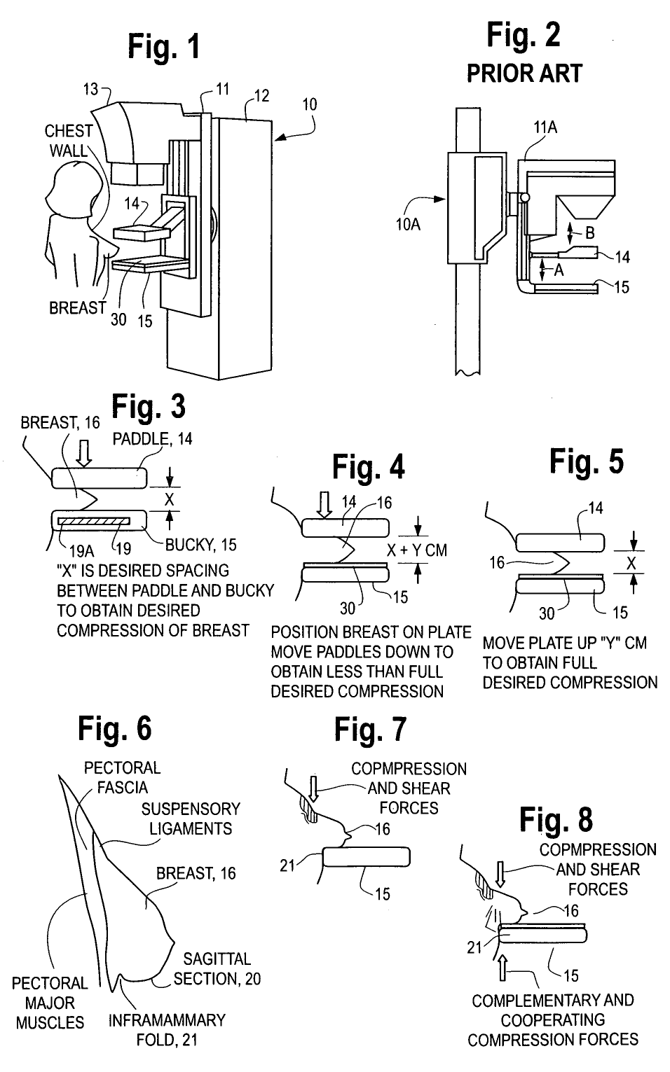

In most mammography machines the bucky 15 is fixedly mounted on the C-arm and is in fixed or stationary position on the C-arm, and only the paddle 14 is movable toward and away from bucky 15. There is however one known X-ray machine that comprises a bucky that is movable on the C-arm relative to the compression paddle. Refer now to FIG. 2 that shows the known machine as disclosed in U.S. Pat. No. 5,590,166 made by Instrumentarium Imaging Inc., now GE Medical Systems Inc. A significant distinction between the machine 10 of FIG. 1 and machine 10A of FIG. 2 is that in machine 10 the bucky is stationary or fixed on the C-arm 11. In contrast, in machine 10A, the bucky is movable on the C-arm 11A. As indicated by the arrow labeled “A” in FIG. 2, the bucky 15 of machine 10A is movable toward and away from the compression paddle 14. Also, as indicated by the arrow labeled “B” the compression paddle 15 is movable toward and away from bucky 15. U.S. Pat. No. 5,590,166 states that machine 10A includes a linear motorized drive to move the compression paddle 14 and the bucky 15 simultaneously in opposite directions at a substantially equal speed for compressing the breast between the compression paddle and the bucky. Machine 10A suggests an action such as that of closing a pair of pliers on a symmetrical object to provide the compression. However, a patient's breast is not a symmetrical object. In contrast to the movement as taught in said patent, the present invention controllably moves the compression paddle and breast interface different distances at different speeds. This compensates for the different structure and sensitivities of the various sections of the breast to thereby reduce pain and discomfort during the compression procedure.

As a modification of the method as described above, the compression paddle 14 and the interface element 30 can be caused to move concurrently, that is, the interface plate could be rising and moving at the same time that the bucky is moving down to compress the breast. However, the amount of movement and the speed of movement of the two components would be different. Moreover, because of the variability of the size, configuration and firmness of the patients' breasts (and the patient's different comfort levels and pain thresholds), concurrent movement appears to be a more critical method.

The inventive method utilizes a movable breast interface element that is mountable on a bucky. The invention also discloses an apparatus including the movable interface element. More specifically, the present invention discloses a breast interface apparatus or assembly 29 including a radiolucent (X-ray transparent) element 30 that is mountable on the bucky 15, see FIG. 1. The interface element 30 functions as a controllably movable breast interface for compressing a patient's breast. Power means are provided for controllably moving the interface element towards (and away) from the compression paddle to practice the inventive method.

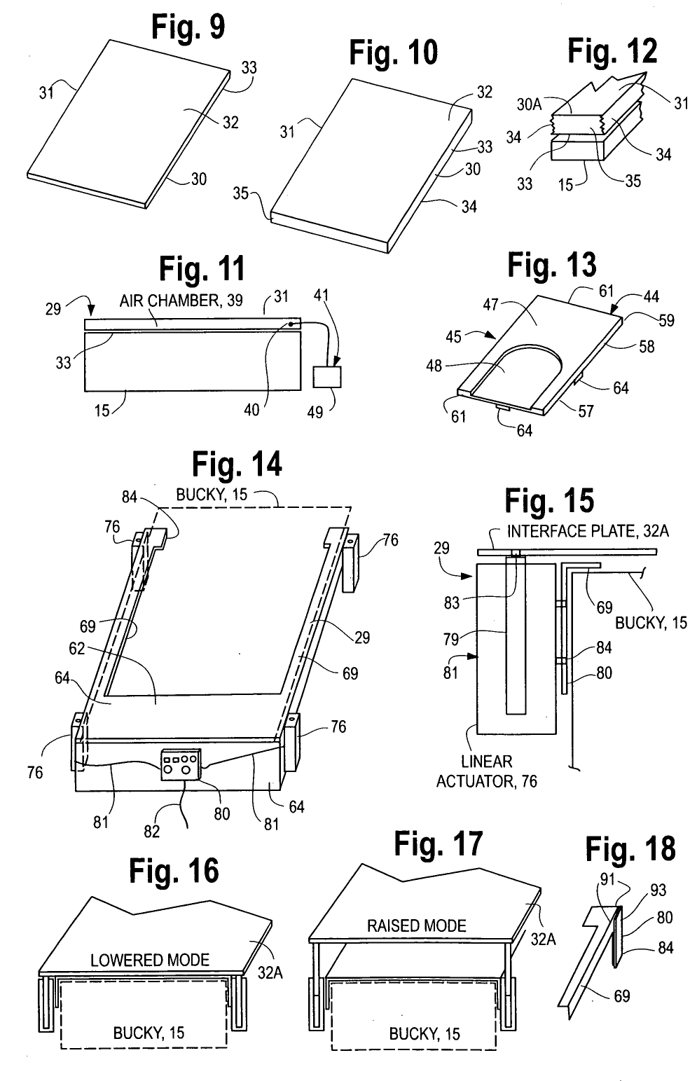

The inventive apparatus will now be further described. Refer to FIGS. 9-18 that show an interface assembly 29 that includes the selectively movable breast interface element 30. The interface element 30, see FIG. 11, is configured for positioning on a bucky 15 to provide an interface between the bucky 15 and a patient's breast 16, see also FIG. 1.

In one embodiment, and referring particularly to FIGS. 9-11, a movable interface element 30 is in the form of an expandable canister 31. The canister 31 includes a top plate 32 (the movable breast interface) and a bottom plate 33, both plates being of radiolucent material. The two plates are joined by sides 34 and ends 35 to form an air chamber 39 between them. The top plate 32 and bottom plate 33 are relatively rigid and the sides are flexible. Canister 31 is inflatable such as by air under pressure received through a suitable two-way valve 40 from an electrically operated air pump 49, of any known design. The canister 31 is shown in a non-inflated mode in FIG. 9 and in an inflated mode in FIGS. 10 and 11.

As can be seen from a comparison of FIGS. 9 and 10, top plate 32 provides a movable surface as required by the inventive method. It is important that the top and bottom plates 32 and 33 be of radiolucent material that will not minimize attenuation of the X-ray beam, such that the imaging process is not adversely affected.

The two-way air valve 40 controls the air admitted to the air chamber 39 to inflate and expand the chamber and move plate 32 upwardly. Air is preferred as the inflating medium. Gases such as helium could be used to provide the pressure to inflate the canister and raise the interface plate 32; however, air is convenient for this purpose. A liquid medium in lieu of air would unsatisfactorily attenuate the X-ray beam.

The valve 39 is coupled to air pump 49 through an air line 43. The air pump may be an electrically operated air pump 49 of any suitable known type that may be coupled to a battery or to an electric outlet. The foregoing types of air pumps, as well as manually operated air pumps, are commercially available. The air provided is at relatively low pressure but it is adequate such that interface plate 32 provides a firm support and firm compression surface. A suitable gauge, not shown, may be provided for pump 49 to monitor the air pressure provided to air chamber 39. Each embodiment of the interface element should provide a force equivalent to the compressive force provided by the compression paddle.

In the embodiment of canister 31 shown in FIGS. 9-12, the sides 34 and ends 35 are made of radiolucent material that is flexible but not stretchable. The top plate 32 and bottom plates 33 each comprise a thin plate of radiolucent material such as PETG (polyethylene terephthalate glycol) plastic having a thickness in the range of 0.075 to 0.125 inch. The PETG material is relatively rigid but it is radiolucent so as to minimally attenuate the X-ray imaging beam.

FIG. 13 shows a canister 44 that is a modification of the canister 32 shown in FIG. 9-12. Canister 44 includes a top plate 45 formed of two sections 47 and 48. One section 48 is made in a partially rounded pattern to accommodate the flattened form of a breast, and is made of flexible material. The other section 47, including the part surrounding the first section 48 is made of relatively rigid material. Sections 47 and 48 are bonded, as by heat-sealing, or glued together. The front end 61 of canister 44 is made of a flexible material and bonded to flexible sides 58, and to the top and bottom plates. The bottom plate 57 of canister 44 is a rigid plastic similar to plate 33 of canister 31. The sides 58 and end 59 of canister 44 are also of rigid plastic similar to plate 33 of canister 31. End 61 is of a flexible material similar to section 48. When canister 44 is in a non-expanded mode, as shown in FIG. 13, section 48 forms a cradle for receiving the patient's breast. When canister 44 is inflated, section 48 is the interface surface that is inflated to form a firm lifting support for the patient's breast.

Tabs 64 formed of relatively rigid plastic may be affixed to the sides 58 and ends of canisters 44 to extend down alongside the bucky. Similar tabs can be provided for canister 31. As is known, the C-arm 11 (on which the bucky is mounted) can be rotated or tilted to take mediolateral oblique images of the breast. Tabs 64 tend to hold the assembly 29 in position on the bucky for this type of oblique procedure. It should be appreciated that canisters and plates of different sizes are provided to accommodate buckys of different dimensions.

The operation or use of the apparatus of FIGS. 9-13 will now be described. Refer to FIG. 11 as well as FIG. 1 the canister 31 is first mounted on the bucky 15. The patient's breast is then positioned on the breast interface plate 32. Next the compression paddle 14 is lowered to engage and compress the patient's breast. As stated above, the compression paddle 14 is moved downwardly toward the bucky 15 to obtain the majority of, but less than the full desired compression. Next, as also described above, movement of the compression paddle 14 is paused. The air pump 49 is then triggered by a switch 41 to provide air under pressure through valve 40 to air chamber 39 to inflate and expand interface plate 32 to a selected height. The air is provided at a relatively slow flow to gently lift the interface plate 32 (in one embodiment, about 5 cm) and to push upwardly against the bottom of the breast to compress the breast. Next, the valve 39 is closed to stop the air flow that stops the movement of the interface plate, as determined by the technician. The breast is now in the proper position for X-ray imaging.

Some “fine adjustment” of the positions of the compression paddle 14 and the interface plate 32 may be required to obtain the exact compression and positioning of the breast. The air valve 40 is a two-way valve that enables a technician to exhaust some air from the air chamber 39 if the compression is more than required, or more than can be tolerated by the patient. The exact amount of movement of the interface plate 32 to obtain the required compression of patient's breast is determined by training and experience. As stated above, the precise or particular relative movement of the paddle 14 and the interface plate 32 varies dependent on the size and configuration of the breast.

The canister 44 of FIG. 13 functions in essentially the same manner as described above. However, in the operation of canister 44, only section 48 expands to move upwardly against a breast cradled therein.

A preferred embodiment of the movable breast interface element is shown in FIGS. 14-18. This latter embodiment of interface element 30A comprises a relatively rigid plastic plate 32A that is radiolucent (X-ray transparent), and is controllably movable by miniature linear actuators 76 such as electric linear actuators or pneumatic air cylinders. Linear actuators of many types are well known in the art and made by many manufacturers. Manufacturers such as the Bimba Manufacturing Co., Monee, Ill. and Parker-Hannifin Corp., Actuator Division, Wadsworth, Ohio make pneumatic air cylinders. Parker-Hannifin Corp. also offers a full line of electric linear actuators as well as miniature pneumatic cylinders. Electric linearly actuators and air cylinders are commonly used in many precise positioning and actuating applications and devices. One common type of electric linear actuator provides linear motion via a motor driven screw assembly.

Refer now to FIG. 14; the interface assembly 29 comprises a generally U-shaped frame 62 that can be mounted on a bucky. The frame 62 has a bight or end 64 that is rectangularly dimensioned to fit over the end of the bucky. The top of bight 64 is supportable on the bucky, and the sides extend alongside the sides of the bucky. The frame 62 includes two arms 69 that are of inverted “L-shape” and extend along the edge of the bucky. The top of the arms 69 is supportable on the top of the bucky and the sides of the arms 69 abut the sides of the bucky. The bight 64 and the arms 69 are dimension so as not to impede the use of slots on the side of the bucky 15 that receive the film cassette or digital imager.

Linear actuators 76 are affixed to the frame 62, two at the ends of arms 69, and two at sides of the bight 64. The actuators may be affixed on support strips 80 that extend downwardly from arms 69, see also FIGS. 15 and 18. In the embodiment of the assembly 29, actuators 76 shown in FIGS. 14 and 15 are electric linear actuators and are powered through suitable leads 81 from a control box 80. Control box 80 is either connected to an electric outlet via lead 82, or is battery powered. FIGS. 15 and 18 also indicate the attachment (by suitable pins or screws 84) of a linear actuator 76 to the strip 80 of arm 69, and the mounting of the arms 69 on the bucky 15. Further, FIG. 15 depicts the coupling or connection 83 of the movable rod 79 of actuator 76 to the interface plate 32A. The connection 83 can be a screw or merely a pin inserted into an open slot in interface plate 32A. The linear actuators 76 controllably raise and lower the interface plate 32A, for the purposes described above.

A movable interface plate 32A that is moved utilizing small components is necessary in view of the limited space available for positioning and operating the interface plate, and because there should be minimal attenuation or interference with the X-ray beam.

As mentioned above, the actuators can also be miniature air cylinders powered by compressed-air pumps or devices that are commercially available from various sources, as mentioned above. The air cylinders are positioned similarly as the electric actuators described above and are actuated by a suitable source 49 of air pressure, as depicted in FIG. 11, to raise and lower the interface plate 32A.

FIG. 16 shows the interface plate 32A in a position adjacent the bucky 15, and FIG. 17 shows the interface plate 32A in a raised or elevated position relative to the bucky 15 to provide compressive forces to the breast.

FIG. 18 shows a support strip 80 formed at the ends of the arms 69. A linear actuator 76 is affixed to each strip 80 by pins or screws 84, as depicted in FIG. 15. Strips 80 are relatively narrow and are formed at the ends of arms 69 to extend downwardly alongside the bucky 15. The positioning of the actuators 76 on the ends of arms 69 does not impede the placing or removing film cassettes or digital imaging accessories through the bucky slot 19A, see FIG. 3. A hinge indicated at 93, in FIG. 18 may be installed and the strip 80 cut on one side and at the top of adjacent arm 69, as at 91, to permit the strip 80 and the affixed linear actuator to swing away from the side of the bucky when inserting or removing film etc.

An advantage of the embodiments shown in FIGS. 15-18 as compared to the embodiment of FIGS. 9-14 is that the interface assembly of FIGS. 15-18 comprises a single radiolucent (X-ray transparent) plate that is mounted in the path of the X-ray beam, whereas the embodiments of FIGS. 9-14 comprise two such plates. A single plate tends to minimize the attenuation of the X-ray beam. As is known, there is some attenuation of the beam, by any material, even those considered as “radiolucent”.

While the invention has been particularly shown and described with reference to preferred embodiments thereof, it will be understood by those skilled in the art that various changes in form and details may be made therein without departing from the spirit and scope of the invention.

1. A method of compressing a patient's breast to a selected compression when taking an image of the breast by a X-ray mammography machine, said machine including a bucky and a compression paddle, said method comprising the steps of,
a) mounting a selectively movable breast interface on said bucky;
b) positioning a patient's breast on said interface:
c) moving said compression paddle toward said interface to compress the breast there between; and
d) moving said interface toward said compression paddle until said selected compression is obtained.

2. A method as in claim 1 including the step of
a) pausing movement of said compression paddle when the compression is less than said selected compression; and then
b) moving said interface toward said compression paddle to provide the selected compression of the breast.

3. A method as in claim 1 wherein said interface comprises a movable plate having a non-elevated mode and an elevated mode, and including the steps of
a) mounting said interface plate when said interface is in a first non-elevated mode; and,
b) moving said interface plate to its elevated mode upwardly against the patient's breast and toward said compression paddle.

4. A method as in claim 1 wherein said interface and said compression paddle are moved at different speeds and for different distances.

5. A method as in claim 1 further including the step of
a) pausing movement of the compression paddle at a position before the full desired compression of the breast is obtained and prior to moving said interface toward said compression paddle.

6. A controllably movable breast interface assembly for mounting on a bucky of a mammography X-ray machine, said machine providing a beam of X-rays for imaging a patient's breast, a breast compression paddle, and a bucky for housing a digital imaging detector or a film cassette, said interface assembly comprising,
a) a radiolucent plate providing an interface between the bucky and a patient's breast;
b) said plate having a top surface for contacting and supporting the patient's breast and a bottom surface mountable adjacent the bucky;
c) linear actuators mounted on said bucky and outside the path of said X-ray beam, said actuators connected to raise and lower said plate relative to said bucky; and
e) means for controllably energizing said actuators for selectively raising and lowering said plate.

7. An interface assembly as in claim 6 further including a canister;
a) said interface plate forming the top part of said canister; and
b) an air chamber formed in said canister for receiving air under pressure to inflate said canister and move said interface plate.

8. An interface plate as in claim 6 wherein said plate includes a flexible breast support section that forms a cradle for the patient's breast when said interface plate is in its non-inflated mode.

9. An interface assembly plate as in claim 6 wherein
a) said interface plate includes tabs depending from said plate;
whereby said tabs tend to maintain said interface plate in position on the bucky.

10. A breast interface assembly for mounting on the bucky of a mammography machine said machine having a C-arm member, the upper arm of said member supporting an X-ray source providing a beam of X-rays, the lower arm of said member supporting a relatively fixed bucky, a compression paddle mountable on the bight portion of the C-arm to be movable thereon, said bucky having a breast supporting surface, and said compression paddle being movable downwardly toward said bucky to compress a patient's breast supported by said bucky, said interface assembly comprising,
a) a breast interface positionable on said bucky;
b) linear actuator means supportable on said bucky, said linear actuators connected to controllably move said interface toward and away from said compression paddle; and
c) a framework for supporting said actuator means on said bucky, said framework being mountable outside the path of said X-ray beam.

11. Apparatus as in claim 10 wherein said linear actuator means comprises an electric linear actuator.

12. Apparatus as in claim 11 wherein said linear actuators are battery powered.

13. Apparatus as in claim 10 wherein said linear actuator means comprises pneumatic air cylinders.

14. Apparatus as in claim 10 wherein said linear actuators are powered from electric outlets.
